(19) United States
(12) Patent Application Publication
(10) Pub. No.: US 2006/0015057 A1
(43) Pub. Date: Jan. 19, 2006

(54) Coating for arterial-venous blood tubing set for hemodialysis system

(76) Inventors: Chih-Hu Ho, Farmington, UST (US); Eric W. Stroup, North Ogden, UT (US); Olli Tuominon, Marlboro, MA (US)

Correspondence Address: FISH & RICHARDSON PC, P.O. BOX 1022, MINNEAPOLIS, MN, 55440-1022, US

(21) Appl. No.: 10/891,882

(22) Filed: Jul. 15, 2004

(51) Int. Cl. A61M 37/00; A41D 19/00

(52) U.S. Cl. 604/6.16; 427/2.3

(57) ABSTRACT

A blood tubing set such as an arterial-venous blood tubing set used in hemodialysis is coated with a copolymer having a hydrophobic segment and a hydrophilic segment. For example, the coating can be PEO-PPO-PEO triblock copolymer. The hydrophobic segment attaches to the tubing set, and the hydrophilic segment prevents or reduces unwanted sorption on the blood tubing set.

### FIELD OF THE INVENTION

The present invention relates to the field of hemodialysis and related medical procedures. More particularly, it relates to coating arterial-venous blood tubing sets used in hemodialysis systems or similar systems.

### BACKGROUND

During hemodialysis, a patient's blood is passed through a dialyzer located outside the patient's body. The procedure is generally used for a patient whose kidneys fail to remove unwanted substances from the patient's blood. During dialysis, the patient's blood is cycled through a hemodialysis system so that unwanted substances are filtered from blood. Additionally (or instead of), desirable substances may be added to the blood. Dialysis patients typically undergo the process several times a week, so the equipment used should be robust.

Hemodialysis is described herein, but it will be appreciated that the disclosed invention may be used in connection with any medical treatment involving removing or introducing fluid into a patient's body, and any such process is deemed to be similar to hemodialysis herein. Examples of such other treatments are hemoperfusion and hemodiafiltration. Similar processes are not limited to treatments involving blood.

In hemodialysis or similar processes, the patient's blood is removed and replaced via a arterial-venous blood tubing set. The blood tubing set must be biocompatible. Still, even with a conventional biocompatible material, blood clotting can cause problems. Products and methods have been developed to mitigate these problems, including regulating patients' diets and using drugs. Nevertheless, a blood tubing set with improved operating characteristics is a welcome advance in the art. The present invention provides an improved blood tubing set.

Patent application Ser. No. 10/013,323 titled Copolymer Coating for a Hydrophobic Membrane, filed on Dec. 7, 2001, owned by the owner of this invention, is hereby incorporated by reference.

### SUMMARY

In the present invention, arterial-venous blood tubing set components are coated. The blood tubing set may be made of polyvinyl chloride (PVC) based materials, and may also include components of polyethylene (PE) or polypropylene (PP) material. The blow tubing set may also be made of other biocompatible materials.

In a preferred embodiment, the coating is performed by pumping 0.2% (w/v) Pluronic F108 (BASF, Mount Olive, N.J.) surfactant through an arterial-venous blood tubing set. Pluronic F108 is well-known surfactant, and the present invention includes generic equivalents and other copolymer coatings. Pluronic F108 has a structure of (PEO)₁₂₉-(PPO)₅₆-(PEO)₁₂₉; where PEO=poly(ethylene oxide) and PPO=poly(propylene oxide). More generally, the present invention relates to coating using copolymers of which Pluronic F108 is a species. In a preferred embodiment the Pluronic F108 is dissolved in water, but other carriers could conceivably be used.

In a preferred embodiment, the coating is applied by circulating the Pluronic F108 surfactant solution through the blood tubing set for 30 minutes, followed by one hour of rinsing the set with RO DI (reverse osmosis deionized) water, followed by drying with compressed air.

### BRIEF DESCRIPTION OF THE DRAWINGS

FIGS. 1-5 show ESCA spectra of coated and uncoated samples of a blood tubing set according to an embodiment of the invention.

FIG. 6 shows a sample of a blood tubing set treated according to an embodiment of the invention.

### DETAILED DESCRIPTION

The present invention involves coating a blood tubing set for use in hemodialysis or related medical procedures. In a preferred embodiment, the arterial-venous blood tubing set is coated, but it is possible to coat just a portion of a blood tubing set if that is desired. As used herein, “blood tubing set” refers to any portion thereof.

The coating modifies the surface to reduce unwanted sorption on the blood tubing set interior surface, and thereby reduce the chance of unwanted reactions. The coating substance uses copolymers with at least one hydrophobic segment and one hydrophilic segment. In a preferred embodiment, polyethylene oxide (PEO) and polypropylene oxide (PPO) copolymers are immobilized on the blood tubing set, coating the surface. The PPO segments are hydrophobic and attach to the surface of the blood tubing set. The blood tubing set, such as a PVC material, is hydrophobic (although that is not critical to the invention, which also includes a hydrophilic blood tubing set or a combination of hydrophobic and hydrophilic). The PEO segments are hydrophilic and do not attach to the blood tubing set, but instead extend into a hydrophilic environment. This helps minimize surface-induced thrombosis reaction or other unwanted reactions between the blood tubing set and the liquids carried within.

The copolymer coating can be applied to the blood tubing set by exposing it to a solution of the copolymers dissolved in water. For example, a PEO-PPO-PEO copolymer can be dissolved in water to form the copolymer solution. This solution can then be transported through the blood tubing set to expose the solution to the set, and the PEO-PPO-PEO copolymers attach to and coat the surface of the blood tubing set.

FIG. 6 shows generally an arterial-venous blood tubing set of the type used with the present invention. Tubing 1 connects to a dialyzer (not shown). Tubing 2 contacts with a blood pump head. Tubing 3 is an air trap on the arterial tubing. Tubing 4 is the filter inside the air trap of the venous tubing. Tubing 5 is the air trap of the venous tubing. It should be understood that these samples are exemplary only, and the invention may be used with any tubing set (including any portion of a tubing set) used in connection with removing or introducing fluids into a patient's body.

In a preferred embodiment, the coating is performed by pumping 0.2% (w/v) Pluronic F108® (BASF, Mount Olive, N.J.) surfactant through an arterial-venous blood tubing set. More generally, the method includes exposing the surfactant to an interior of at least a part of a blood tubing set. Pluronic F108 is a well-known surfactant, and the present invention includes generic equivalents and other copolymer coatings. Pluronic F108 has a structure of (PEO)₁₂₉-(PPO)₅₆-(PEO)₁₂₉; where PEO=poly(ethylene oxide), PPO=poly(propylene oxide). More generally, the present invention relates to coating using tri-block copolymers of which Pluronic F108 is a species. Other triblock copolymers include (PEO)₇₆-(PPO)₃₀-(PEO)₇₆ and (PEO)₁₀₄-(PPO)₃₉-(PEO)₁₀₄.

The weight/volume of the solution should be less than the critical gel point so the solution can easily flow through the tubing set. The critical gel point for a solution of Pluronic F108 is approximately 3% (weight/volume). During the coating process, the solution must be kept above freezing, and it is believed that the coating process will work well when the solution has a temperature greater than 20 degrees C. The process works particularly well when the solution is maintained at about 37 degrees C.

In a preferred embodiment, the coating is applied by circulating the Pluronic F108 surfactant for 30 minutes, followed by one hour of rinsing the set with RO DI water, followed by drying with compressed air. Commercially available PEO-PPO-PEO triblock copolymers are powdered substances that can be dissolved in water to accomplish the circulating step.

Other copolymers have a hydrophobic segment and a hydrophilic segment. For example, other PEO-PPO-PEO triblock copolymers can be used in the invention. From a general point of view, the present invention involves, in one aspect coating hydrophobic surface to from a hydrophilic surface, and in a more general aspect, coating a biocompatible blood tubing set with a triblock copolymer.

This above description enables a blood tubing set having a coating of a copolymer that has a hydrophobic segment and a hydrophilic segment adhered to an inner surface of tubing. It will be appreciated that the present invention can be used with other processing steps, if desired.

### EXPERIMENTAL RESULTS

Electron spectroscopy for chemical analysis (ESCA) is generally regarded as a key technique for the surface characterization and analysis of biomedical polymers. This technique provides a total elemental analysis of the top 10 to 200 Angstroms of the surface. The basic principle of ESCA is the photoelectric effect. Qualitative analysis of the ESCA results are presented below. The control refers to a test of an untreated sample. The coated entry refers to a sample treated according to the preferred embodiment. Each tubing sample was a cut to a 1 cm length. All the samples tested were the inner surface of the tubing except, sample #4 tested the outside surface of the sample.

TABLE 1

| | | | Control Atom | Coated Atom |
|---|---|---|---|---|
| Sample | Atom | | % | % |
| Sample 1 | O | 1s | 9.75 | 10.52 |
| | C | 1s | 83.26 | 84.26 |
| | Cl | 2p | 6.99 | 5.22 |
| Sample 2 | O | 1s | 9.87 | 10.84 |
| | C | 1s | 87.97 | 85.3 |
| | Cl | 2p | 2.16 | 3.85 |
| Sample 3 | O | 1s | 3.37 | 4.81 |
| | C | 1s | 71.10 | 70.73 |
| | Cl | 2p | 25.53 | 24.46 |
| Sample 4 | O | 1s | 0.98 | 4.89 |
| | C | 1s | 99.02 | 95.11 |
| Sample 5 | O | 1s | 6.86 | 6.33 |
| | C | 1s | 73.67 | 73.02 |
| | Cl | 2p | 19.46 | 20.65 |

Samples 1, 2, 3, and 5 are PVC based materials. Sample 4 is a polyethylene (PE) or polypropylene (PP) based material. All the PVC samples had trace impurity elements such as Zn, Ca, etc., which probably came from the PVC plasticizer. The major elements are C, O, and Cl in PVC, and C in PE (or PP). Since C and O are the only elements in Pluronic F108, determination of the chemical composition of the coating focuses on the total oxygen element percentage changes. Oxygen content increased in samples 1, 2, and 3, and increased greatly in sample 4. (The oxygen content decreased slightly for sample 5). These results indicate that the tubing set was coated by the Pluronic F108.

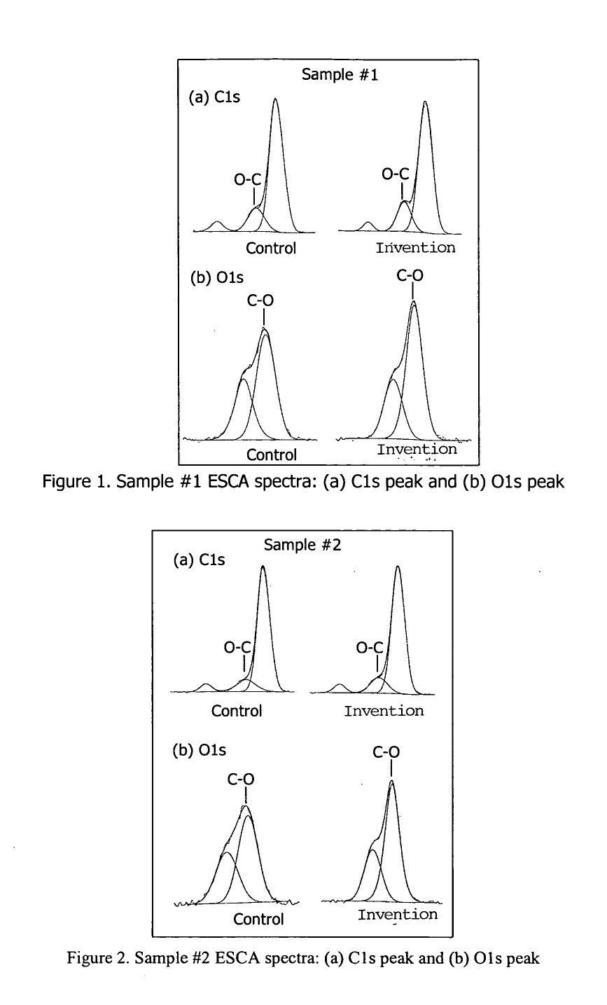

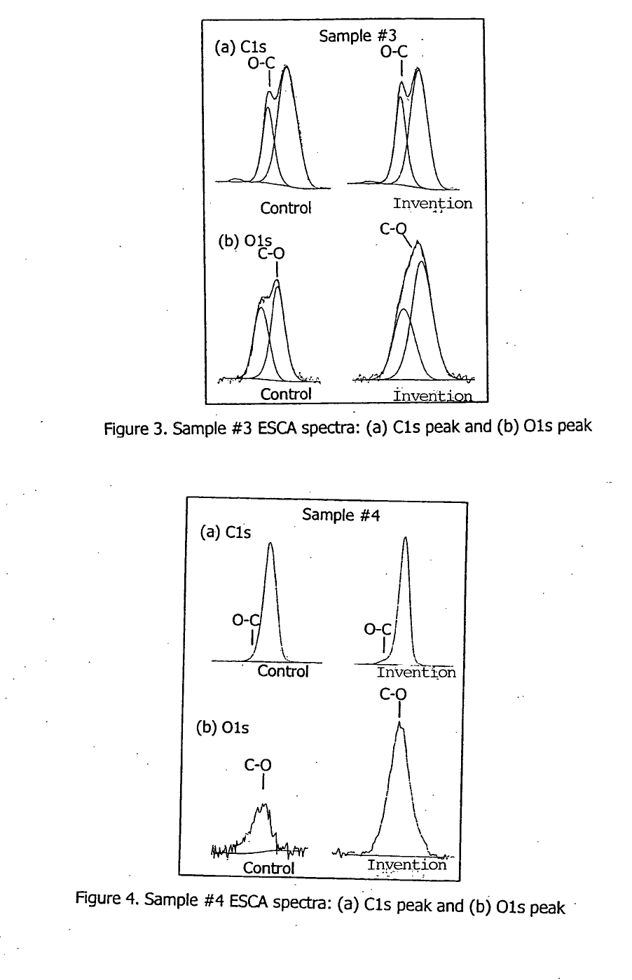

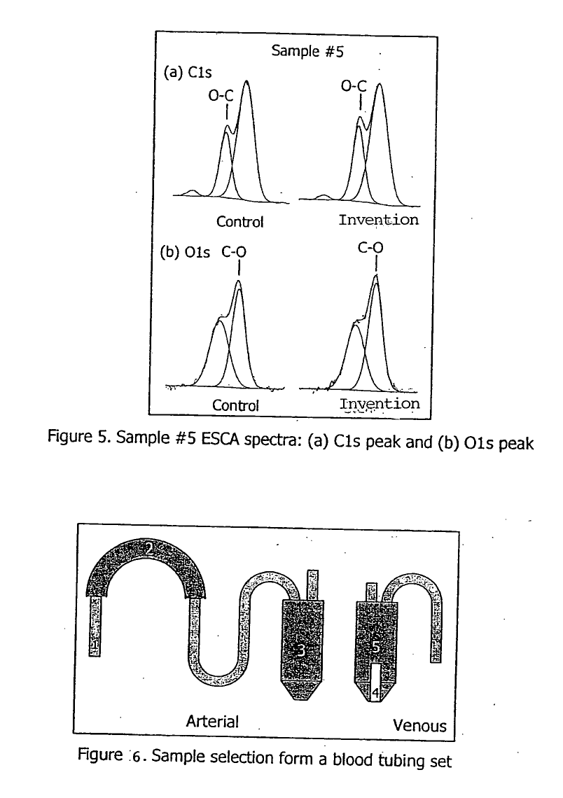

Since oxygen is the key element, a discussion of the ESCA spectrum focuses on the C—O bond changes in both the C1s and O1s spectrum. FIGS. 1-5 show the spectra of the sample. Each figure contains (a) C1s and (b) O1s peaks for both control and coated blood tubing sample. Each figure is numbered to correspond to the like-numbered tubing sample.

Compared to the control samples, the O—C bond peaks for all of the C1s spectra show slight increases. This change due to the O—C bond describes only a small portion of the entire C contents of the surface. Based on the chemical structure of the bulk materials of PVC and PE (or PP), the C—C bond dominates the majority of the entire C1s peak. On the other hand, the C—O bond is the majority in the entire O1s peak due to less oxygen contents in the bulk materials. In FIG. 4, there is a small CO bond tail on the coated C1s spectra and a great increase of the C—O bond peak on the coated O1s peak. All these peak intensity changes indicate that the coating has been bound to the blood tubing set.

What is claimed is:

1. A method for coating a blood tubing set useful for hemodialysis or a related procedure, the method comprising exposing the blood tubing set to a copolymer having a hydrophobic segment and a hydrophilic segment.

2. The method of claim 1, wherein the hydrophobic segment is PPO and the hydrophilic segment is PEO.

3. The method of claim 2, wherein the copolymer is of (PEO)₁₂₉-(PPO)₅₆-(PEO)₁₂₉.

4. The method of claim 1, wherein the exposing step includes using a solution of about 0.2% (w/v) of (PEO)₁₂₉-(PPO)₅₆-(PEO)₁₂₉.

5. The method of claim 4, wherein the exposing is done for about 30 minutes.

6. The method of claim 5, further comprising the steps of, after exposing, rinsing the blood tubing set with RO DI water, followed by drying with compressed air.

7. The method of claim 1, wherein the exposing step includes exposing the copolymer to a hydrophobic material.

8. The method of claim 1, wherein the exposing step includes exposing the copolymer to both a hydrophobic material and hydrophilic material.

9. A coated blood tubing set made by the process comprising exposing a blood tubing set to a copolymer having a hydrophobic segment and a hydrophilic segment.

10. A coated blood tubing set according to claim 9, wherein the hydrophobic segment is PPO and the hydrophilic segment is PEO.

11. A coated blood tubing set according to claim 9, wherein the copolymer is (PEO)₁₂₉-(PEO)₅₆ (PEO)₁₂₉.

12. A coated blood tubing set according to claim 9, wherein the exposing step includes using a solution of about 0.2% (w/v) of (PEO)₁₂₉-(PPO)₅₆-(PEO)₁₂₉.

13. A coated blood tubing set according to claim 12, wherein the exposing is done for about 30 minutes.

14. A coated blood tubing set according to claim 13, further comprising the steps of, after exposing, rinsing the blood tubing set with RO DI water, followed by drying with compressed air.

15. A coated blood tubing set according to claim 9, wherein the exposing includes exposing the copolymer to a hydrophobic material.

16. A coated blood tubing set according to claim 9, wherein the exposing includes exposing the copolymer to a both a hydrophobic material and a hydrophilic material.

17. A blood tubing set comprising a coating of a copolymer having a hydrophobic segment and hydrophilic segment adhered to an inner surface of a tubing.
